(19) United States
(12) Patent Application Publication
(10) Pub. No.: US 2021/0244791 A1
(43) Pub. Date: Aug. 12, 2021

(54) APPLICATION OF PEDF-DERIVED SHORT PEPTIDES IN TENDON HEALING

(71) Applicant: BRIM Biotechnology, Inc., Taipei (TW)

(72) Inventors: Frank Wen-Chi Lee, Bedford, MA (US); Yuan-Ming Lee, Taipei (TW); Yeou-Ping Tsao, Taipei (TW); Tsung-Chuan Ho, Taipei (TW)

(73) Assignee: BRIM Biotechnology, Inc., Taipei (TW)

(21) Appl. No.: 17/053,047

(22) PCT Filed: Apr. 8, 2019
(86) PCT No.: PCT/US2019/026307

§ 371 Date: Nov. 4, 2020

Related U.S. Application Data
(60) Provisional application No. 62/654,466, filed on Apr. 8, 2018.

(51) Int. Cl. A61K 38/16; A61K 38/10

(52) U.S. Cl. CPC A61K 38/16; A61K 38/10

(57) ABSTRACT

A method for treating a tendon injury includes administering to a subject in need thereof a pharmaceutical composition comprising a PEDF-derived short peptide (PDSP) or a variant of the PDSP, wherein the PDSP comprises residues 93-106 of human pigmented epithelium-derived factor (PEDF), and wherein the variant of the PDSP contains serine-93, alanine-96, glutamine-98, isoleucine-103, isoleucine-104, and arginine 106 of the PDSP and contains one or more amino acid substitutions at other positions, wherein residue location numbers are based on those in the human PEDF.

### FIELD OF THE INVENTION

This invention relates to PEDF-derived peptides and their uses in tendon healing after injuries.

### BACKGROUND OF THE INVENTION

Tendons contain dense connective tissues and execute the transmission of muscle force to bone, which is crucial to the control of body movement. Tendon injuries are common and often caused by overstretching the tendon. However, tendon has limited ability of self-healing after severe injury because of its avascularity and acellularity. Unlike other type of connective tissues, it is difficult to mobilize bone marrow mesenchymal stromal cells (BM-MSCs) to the injury site of tendon. The repair of tendon is thus a slow and relatively difficult process.

Recently, intensive efforts have been made to employ cell therapy-based approaches to accelerate tendon regeneration and repair. Adult MSCs can be obtained to provide adequate cell source for tendon regeneration. However, cell transplantation requires a time-consuming expansion process. In addition, costs, technical and safety issues are hurdles in providing benefits to patients. Growth factor that stimulates tendon stem/progenitor cells (TSPC) proliferation may be an alternative option to promote tendon repairs. Recent work has shown that TSPC carrying the CD146 marker at tendon periphery can be stimulated to proliferate in tendon wound after providing connective tissue growth factor (CTGF) locally. Platelet-rich plasma (PRP) injection is another example for harvesting the potential of platelet derived growth factor (PDGF) for tendon injury healing, although the effect is limited, possibly due to low concentration of PDGF in the preparation. Moreover, growth factor treatment is readily available for acute tendon injury, skipping the waiting period of cell therapy.

Several growth factors have been reported to possess abilities to induce TSPC proliferation. Connective tissue growth factor (CTGF) has been demonstrated to enhance the clonogenic capacity of CD146+TSPC. Fibroblast growth factor (FGF)-2 promotes growth of TSPC marked by Scleraxis (Scx) and SRY-box containing gene 9 (Sox9) expressions. In addition, it has been reported that hydrogel combinations of bFGF, insulin-like growth factors (IGF)-1, and PDGF-BB can improve the survival of adipose-derived mesenchymal stem cells (ASCs) to assist tendon healing in vivo.

Pigmented epithelium-derived factor (PEDF) is widely expressed in most body tissues. PEDF has been reported to mediate several stem/progenitor cell proliferations. For example, PEDF is effective in stimulating the proliferation of neuronal progenitor cells and human embryonic stem cells. Recent studies further demonstrated that PEDF and PEDF-derived short peptides (PDSP) can stimulate the proliferation of limbal stem cells, muscle satellite cells, and hepatic stem cells. These observations suggest that PDSP may be a therapeutic agent with promising potential for several types of tissue injuries, including tendon injuries.

### SUMMARY OF INVENTION

One aspect of the invention relates to pharmaceutical compositions or methods for treating tendon injuries. A method in accordance with one embodiment of the invention includes administering to a subject in need thereof a pharmaceutical composition comprising a PEDF-derived short peptide (PDSP) or a variant of the PDSP, wherein the PDSP comprises residues 93-106 of human pigmented epithelium-derived factor (PEDF), and wherein the variant of the PDSP contains serine-93, alanine-96, glutamine-98, isoleucine-103, isoleucine-104, and arginine 106 of the PDSP and contains one or more amino acid substitutions at other positions, wherein residue location numbers are based on those in the human PEDF. The PDSP comprises the sequence of the sequences of any one of SEQ ID NO: 1 to 75.

Other aspect of the invention will become apparent with the following detailed description and the attached claims.

### BRIEF DESCRIPTION OF THE DRAWINGS

FIG. 1 shows the effects of 29-mer variants on nucleostemine-positive TSPC proliferation. Primary rabbit TSPC were cultured to near-confluence in a 75T cell culture flask and then verified by immunostaining of a TSPC marker, nucleostemin (>98%; green color). Nuclei were stained with Hoechst 33258 (blue color) and visualized by an epifluorescence microscopy. The nucleostemine-positive TSPC were treated with 10 μM 29-mer or its variants for 24 h. The numbers of TSPC after 24-h expansion were detected with a cell proliferation assay kit (BioVision; catalog number: K307). The TSPC treated with PDSP solvent was set as 100%. Results are expressed as mean±SE of three independent experiments.

FIG. 2 shows results of western blot analysis of the expression of cyclin D1 in TSPC treated with the 29-mer variants. Primary rabbit TSPC were treated with 10_{N}M 29-mer or its variants for 24 h. Representative blots (A) and densitometric analysis with the SD (B) from three independent experiments are shown. Cyclin D1 expression was normalized to (3-actin. *P<0.01 vs. solvent-treated cells. ^{#}P<0.05 vs. 29-mer-treated cells.

FIG. 3 shows histological appearance of the 29-mer variant-treated tendons after 1 week postoperative. Representative micrographs of the histopathological analysis by H&E staining. H&E-stained sections in higher magnification show uninjured tendon tissue with a relative scarcity of cells among the collagen fibers. The injured region shows degeneration and inflammation marked by yellow arrows to indicate fatty deposits, black Arrows to indicate a zone with high cellularity and * to indicate vessels.

FIG. 4 shows the effect of 29-mer variant/alginate gel on Achilles tendon healing. (A) Representative H&E-stained longitudinal sections in higher magnification show the nuclei morphologies in uninjured tendon tissue and injured tendon treated with vehicle/alginate and 29-mer/alginate for 1 week. (B) Histopathological scores. Total scores were determining by fiber structure, fiber arrangement, rounding of the nuclei, resident cell density and inflammation (infiltration of inflammatory cells, neovascularization and fatty deposits). Data are reported as mean±SE. *P<0.0005 versus vehicle/alginate-treated tendon; ^{#}P<0.05 versus 29-mer/alginate-treated tendon.

FIG. 5 shows the effect of 29-mer variant/alginate gel on CD146-positive TSPC expansion in injured Achilles tendon. (A) Representative CD146-stained longitudinal sections. (Original magnification×200). (B) The number of CD146-positive TSPC per 200×field of view on injured tendon sections. Data are reported as mean±SE. Total CD146⁺ cells were evaluated from 6 sections/tendon specimen, with 3 rats in each group. *P<0.001 versus vehicle/alginate-treated tendon; #p <0.001 versus 29-mer/alginate-treated tendon.

### DETAILED DESCRIPTION

Embodiments of the invention relates methods for treating tendon injuries using PEDF-derived short peptides (PDSP). Human Pigment Epithelium-derived Factor (PEDF) a secreted protein containing 418 amino acids, with a molecular weight of about 50 kDa. PEDF is a multifunctional protein with many biological functions (see e.g., U.S. Patent Application Publication No. 2010/0047212). Different peptide regions of the PEDF are found to be responsible for different functions. For example, a 34-mer fragment (residues 44-77 of PEDF) has been identified to have anti-angiogenic activity, while a 44-mer fragment (residues 78-121 of PEDF) has been identified to have neurotrophic properties.

Inventors of the present invention found that certain short peptides of PEDF can be used to treat tendon injuries. It was further found that the therapeutic effects may arise from the abilities of these PDSPs to induce CD146+ TSPC expansion. CD146⁺ TSPC distributes at peripheral region of rat tendon and CD146⁺ TSPC has been found to assist wound healing of rat patellar tendon.

The PDSPs of the invention are based on the peptide region corresponding to human PEDF residues 93-121 (⁹³SLGAEQRTESIIHRALYYDLISSPDIHGT¹²¹; SEQ ID NO:1). Based on this 29-mer, inventors identified that serine-93, alanine-96, glutamine-98, isoleucine-103, isoleucine-104, and arginine-106 are critical for the activities, as evidenced by significant loss of activities when these residues were individually replaced with alanine (or glycine for Alanine-96). In contrast, alanine (or glycine) replacements of other residues in the 29-mer did not significantly change the activities, suggesting PDSP variants having amino acid substitutions (particularly, homologous amino acid substitutions) at these other residues (i.e., residues 94, 95, 97, 99-102, 105, and 107-121) can also be used to prevent and/or treat tendon injuries.

These results indicate that the core peptide containing the antinociceptive effects is in the region comprising residues 93-106 (⁹³SLGAEQRTESIIHR¹⁰⁶; SEQ ID NO:2). Thus, the shortest PDSP peptide having the therapeutic activity for tendon injuries may be a 14-mer. One skilled in the art would appreciate that addition of additional amino acids to this core peptide, at the C and/or N terminus, should not affect this activity. That is, a PDSP of the invention may be any peptide comprising residues 93-106 (-) of human PEDF. Therefore, a PDSP peptide for the invention may be a 14-mer, 15-mer, 16-mer, and so on, including the 29-mer used in the experiments.

Furthermore, as noted above, substitutions within these short peptides can retain the activities, as long as the critical residues (serine-93, alanine-96, glutamine-98, isoleucine-103, isoleucine-104, and arginine-106) are preserved. In addition, the mouse variants (which have two substitutions: histidine-98 and valine-103, as compared with the human sequence) are also active. The corresponding mouse sequences are: mo-29mer (SLGAEHRTESVIHRALYYDLITNPDIHST, SEQ ID NO: 3) and mo-14mer (SLGAEHRTESVIHR, SEQID NO: 4). Thus, a generic sequence for an active core is (93S-X-X-A-X-Q/H-X-X-X-X-I/V-I-X-R¹⁰⁶, wherein X represents any amino-acid residue; SEQ ID NO: 5).

PDSP peptides of the invention may be chemically synthesized or expressed using protein/peptide expression systems. These PDSP peptides may be used in a pharmaceutical composition for the treatment of tendon injuries. The pharmaceutical composition may comprise any pharmaceutically acceptable excipient, and the pharmaceutical composition may be formulated in a form suitable for administration, such as topical application, oral application, injection, etc. Various formulations for such applications are known in the art and can be used with embodiments of the invention.

Some embodiments of the invention relate to methods for treating tendon injuries in a subject (e.g., human, pets, or other subjects). As used herein, the term “treat” or “treating” includes partial or total improvement of the condition, which may or may not include total cure. The method may comprise administering a pharmaceutical composition to the subject, wherein the pharmaceutical composition comprises an effective amount of a PDSP of the invention (including active variants of the PDSP). One skilled in the art would appreciate that the effective amount would depend on the conditions of the subject (e.g., weight, age, etc.), the route of administration, and other factors. Finding such effective amount involves only routine techniques and one skilled in the art would not require inventive efforts or undue experimentation to find the effective amount.

Embodiments of the invention will be illustrated with the following specific examples. In specific examples, the 29mer (SEQ ID NO:1) are used. However, other PDSP (e.g., 14mer, SEQ ID NO:2 or SEQ ID NO:3, etc.) can also be used to achieve the same results. One skilled in the art would appreciate that these examples are for illustration only and that variations and modifications are possible without departing from the scope of the invention.

Materials and Methods

Dulbecco's modified Eagle's medium (DMEM), fetal bovine serum (FBS), antibiotic-antimicotic solutions, and trypsin were purchased from Invitrogen (Carlsbad, Calif., USA). 5-Bromo-2′-deoxyuridine (BrdU), insulin-transferring-sodium selenite (ITSE) media supplement, Hoechst 33258 dye, Alginic acid sodium salt, and all chemicals were from Sigma-Aldrich (St. Louis, Mo., USA). Dispase II and collagenase I were obtained from Roche (Indianapolis, Ind., USA). Anti-BrdU antibody (GTX42641) was from GeneTex (Taipei, Taiwan). Anti-nucleostemin (ab70346) antibodies were from Abcam (Cambridge, Mass., USA). All the fluorescent dye-conjugated secondary antibodies were purchased from BioLegend (San Diego, Calif., USA). Hematoxylin and eosin (H&E) dyes were purchased from Merck (Rayway, N.J., USA). The PDSP 29-mer and 29-mer variants were synthesized, modified by acetylation at the NH₂ termini and amidation at the COOH termini for stability, and characterized by mass spectrometry (>90% purity) at GenScript (Piscataway, N.J., USA). Each PEDF-derived synthetic peptide was reconstituted in DMSO as stock (10 mM).

### Animal Studies

All animals were housed in an animal room under temperature control (24-25° C.) and 12:12 light-dark cycle. Standard laboratory chow and tap water were available ad libitum. Experimental procedures were approved by the Mackay Memorial Hospital Review Board (New Taipei City, Taiwan, R.O.C.) and were performed in compliance with national animal welfare regulations.

### Isolation and Culture of Tendon Stem Cells

Achilles tendons from New Zealand White rabbits (6-8 months old, 3.0-4.0 kg) were used in this study. Achilles tendons were washed two times with sterile phosphate-buffered saline (PBS) containing 50 μg/ml gentamicin. The tendon and tendon sheath were cut into small pieces (1-2 mm³). Each 100 mg of fragment was then digested in a solution containing 3 mg/ml of type I collagenase and 4 mg/ml of dispase in 1 ml balance salt solution (BSS; Alcone) at 37° C. for 4 hours. The digested tissues were washed three times with PBS and collected by centrifugation (800 g for 10 min). The digested tissues were placed in tissue-culture plates (Falcon Labware; NJ, USA) and resuspended in high-glucose DMEM supplemented with 10% FBS and 50 μg/ml gentamycin, and maintained at 37° C. with 5% CO₂. After 5 days, the medium was changed to remove the loosened tissue residues. Subsequently, tendon cells were incubated with 10% FBS medium for 2 days, and then cultured with a basal medium (2% FBS, 1% ITSE, 300 μg/ml L-glutamine, 1% antibiotic-antimicotic solutions) for further 10 days. Culture medium was changed every 3 days. For passage, tendon cells were harvested with 0.25% trypsin/EDTA, cell counting by haemocytometer, approximately 5×10³ cells were seeded in each well of a 96-well cell culture plate or 2×10⁵ cells were seeded in each well of a 6-well cell culture plate for 24 h. These expanded tendon cells were then treated with 10 μM 29-mer or its variants in fresh basal medium for further 24 h and subjected to cell proliferation assay and western blot analysis, respectively.

### Cell Proliferation Assay

Cell Proliferation Assay Kit (Fluorometric) was purchased from BioVision (Catalog #: K307) and used to evaluate cell proliferation according to the manufacturer's recommendations. The fluorescence was read at 480 nm for excitation and 538 nm for emission on a SPECTRAmax GEMINI XS fluorescence microplate spectrophotometer (Molecular Devices, Sunnyvale, Calif., USA).

### Immunocytochemistry

Cells were fixed with 4% paraformaldehyde and then treated at 4° C. with methanol for 1 min and blocked with 1% goat serum and 5% BSA for 1 h. Cells were stained with antibodies to nucleostemin (1:100 dilution) at RT for 3 h. The slides were subsequently incubated with the FITC-donkey anti-rabbit IgG (1:500 dilution; BioLegend, San Diego, Calif.) for 20 min and then counterstaining with Hoechst 33258 for 6 min. Slides were rinsed with PBS with Triton X100 (0.5%) for three times. Finally, the sections were mounted with FluorSave reagent (Calbiochem), and viewed with a Zeiss epifluorescence microscope.

### Western Blot Analysis

Cell lysis, SDS-PAGE, and antibodies used for immunoblotting were performed as described (Ho et al., 15-deoxy-Delta(12,14)-prostaglandin J2 induces vascular endothelial cell apoptosis through the sequential activation of MAPKS and p53. J Biol. Chem., 2008; 283(44): 30273-30288). The band intensity in immunoblot was evaluated with a Model GS-700 imaging densitometer (Bio-Rad Laboratories, Hercules, Calif.) and analyzed using Labworks 4.0 software.

### Surgical Procedure for Rat Achilles Tendon Injury

To investigate the effects of the 29-mer peptide on tendon healing, a rat model of Achilles tendon injury was established as reported by Orhan et al. (The effect of extracorporeal shock waves on a rat model of injury to tendon Achilles. A histological and biomechanical study. J. Bone Joint Surg. Br. 2004;.86(4):.613-618). Adult 10-week-old male Sprague-Dawley rats (initial body weight=312±11 g) were anesthetized by an intraperitoneal injection of xylazine (10 mg/kg). Then, the left tendo Achilles injury was created by full-thickness insertion of an 18-G needle through tendo Achilles 1 cm proximal to the calcaneum attachment site. This created a horizontal wound that was flanked by intact tendon tissue to prevent the retraction of severed ends. The skin incision was closed after the wound was irrigated with sterile saline. Treatments were applied to area around the tendon lesion by subcutaneous injection with 150 μl of alginate gel mixed with 100 μM 29-mer or DMSO vehicle (six rats per experimental condition).

### In Vivo Detection of DNA Synthesis

For the detection of cell expansion, BrdU was reconstituted in DMSO as stock (80 mM). 150 μl of BrdU mixed with 350 μl of PBS was intraperitoneally injected into the rat on day 0, 3, 5 after surgery. DNA synthesis was assessed by BrdU labeling with anti-BrdU antibodies.

### Histological Examination of the Tendon Injury

The tendon and surrounding soft tissue were dissected. Specimens were fixed in a 4% paraformaldehyde (PFA) solution and then were embedded in paraffin blocks. Sections (5 μm in thickness) were longitudinally cut and stained with hematoxylin and eosin (H&E) or used for immunohistochemical examination. 36 sections per tendon were carefully prepared so as to include the most severely degenerated area. Images were captured using a Nikon Eclipse 80i microscope (Nikon Corporation, Tokyo, Japan) equipped with a Leica DC 500 camera (Leica Microsystems, Wetzlar, Germany). Four sections of each sample containing injured tendon tissues were selected and evaluated by two blinded observers to assess the tendon morphology according to a modified semi-quantitative grading score from 0 to 3 proposed by Chen et al. (Tendon derived stem cells promote platelet-rich plasma healing in collagenase-induced rat Achilles tendinopathy. Cell Physiol. Biochem., 2014; 34(6): 2153-68). The scores analyzed the fiber structure (0-3), fiber arrangement (0-3), rounding of the nuclei (0-3), resident cell density (0-3), and inflammation (0-3; infiltration of inflammatory cells, neovascularization and fatty deposits). According to this grading system, a perfectly normal tendon obtained a score of 0, whereas a score of 15 was assigned to maximally abnormal tendons.

### Immunohistological Staining

Formalin-fixed, paraffin-embedded tendon specimens were deparaffinized in xylene and rehydrated in a graded series of ethanol concentrations. Slides were blocked with 10% goat serum for 60 min and then incubated with primary antibody against CD146 (1:50 dilution) at room temperature (RT) for 2 h. The slides were subsequently incubated with the appropriate peroxidase-labeled goat immunoglobulin (1:500 dilution; Chemicon, Temecula, Calif.) for 20 min and then incubated with chromogen substrate (3,3′-diaminobenzidine) for 2 min before counterstaining with hematoxylin.

### Statistics

Results were expressed as the mean±standard error of the mean (SEM). 1-way ANOVA was used for statistical comparisons. P<0.05 was considered significant, unless otherwise specified.

### Identification of Critical Amino Acid Residues in the 29-Mer Required for the Mitogenic Activity to Induce TSPC Proliferation in Culture

We showed that fragments of pigment epithelium-derived factor (PEDF)-derived short peptides (29-mer; residues Ser93-Thr121, 24-mer; Ser93-Pro, and 20-mer; Ser93-Leu112) facilitated healing of tendon rupture as evidenced by a rat model. The results of the previous study also indicated that 29-mer, 24-mer and 20-mer added in culture medium were able to induce cultured tendon stem/progenitor cells (TSPC) proliferation. Here, single residue substitutions with alanine or glycine along the 29-mer sequence were designed and synthesized to elucidate the critical residues responsible for the 29-mer mitogenic activity. In total, 29 peptides variants were synthesized based on the amino acid sequence of PEDF residues 93-121, including 27 with a single alanine alteration and 2 with a single glycine alteration (A96G and A107G). Firstly, we investigated the effects of the 29-mer variants on the proliferation of TSPC.

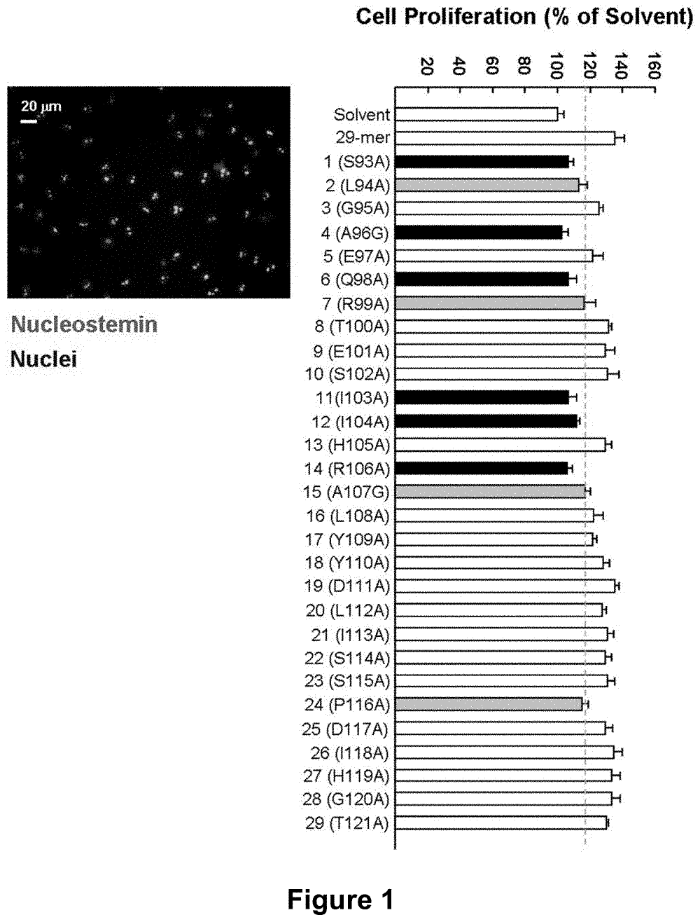

Rabbit TSPC isolation was described above. TSPC in low serum media were treated with 10 μM of one of the 29-mer variants for 24 h. Cell proliferation was examined by a cell proliferation kit based on a kit provided nuclear dye that specifically binds to nucleic acid in the cell and generates green fluorescence. 29-mer treatment increased TSPC proliferation, as compared to DMSO solvent control (135±6.1% versus 100±4.0%, FIG. 1). The results also revealed that S93A (106±3.8%), A96G (102±4.0), Q98A (106±5.4%), 1103A (106±5.3%), 1104A (112±1.7%), and R106A (106±3.5%) mutations severely impaired the mitogenic activity of the 29-mer (102˜112% versus 135%). These results suggest that 6 out of 29 amino acids are critical for the 29-mer activity. In addition, L94A (113±5.2%), R99A (116±7.0), A107G (117±3.8%), and P116A (115±3.7%) mutations caused partially reductions in the mitogenic activity of 29-mer to 112˜117%. The remaining substitutions preserved more than half of the mitogenic activity, compared to the 29-mer (>117%). These results suggest that the remaining 19 residues in the 29-mer can tolerate amino acid substitutions.

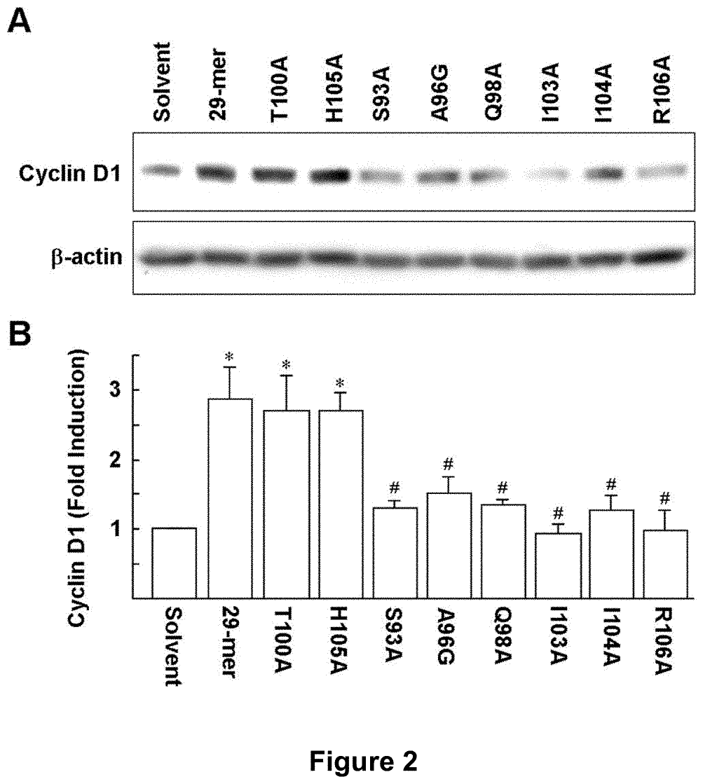

Meanwhile, exposure of TSPC to the 29-mer and its variants for 24 h resulted in a 2.9±0.5-fold induction of the cyclin D1 protein, a proliferative marker, as compared with solvent-treated cells. T100A and H105A variants showed similar effect as the 29-mer to induce cyclin D1 protein expression (2.7±0.5 and 2.7±0.3-fold; FIG. 2). However, the 29-mer effect on cyclin D1 protein induction was almost abolished by alanine/glycine replacement at residue S93, A96, Q98, 1103, 1104 and R106, respectively (1.3±0.1, 1.5±0.3, 1.3±0.1, 0.9±0.1, 1.3±0.2, and 1.0±0.3). Collectively, alanine scanning data indicate that the mitogenic effect of the 29-mer on TSPC is influenced by the amino acid substitutions. The data also imply that the PDSP at positions Ser93, Ala96, Gln98, Ile103, Ile104, and Arg106 are important for sustaining the PDSP effect on induction of TSPC proliferation.

### Therapeutic Effect of the 29-Mer Variants in Rat Model of Experimental Tendon Injury

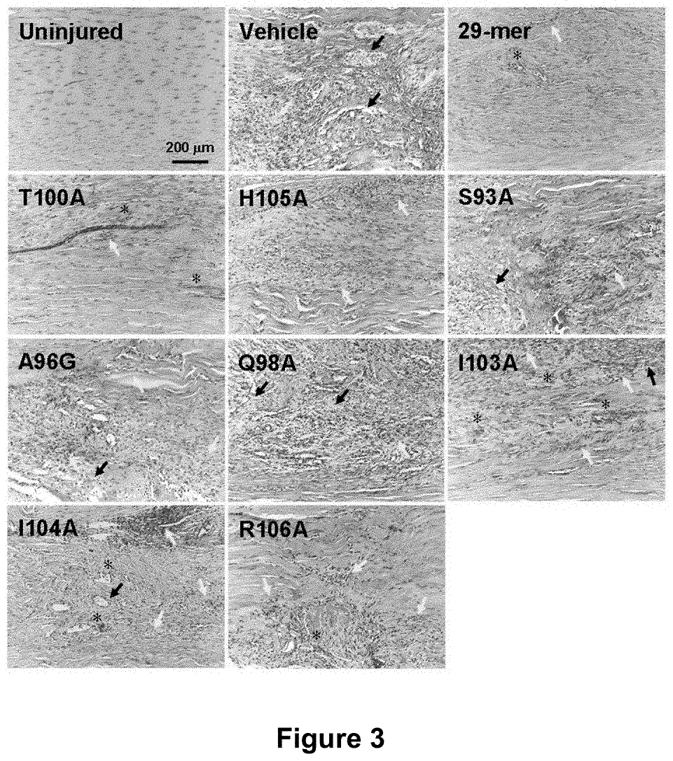

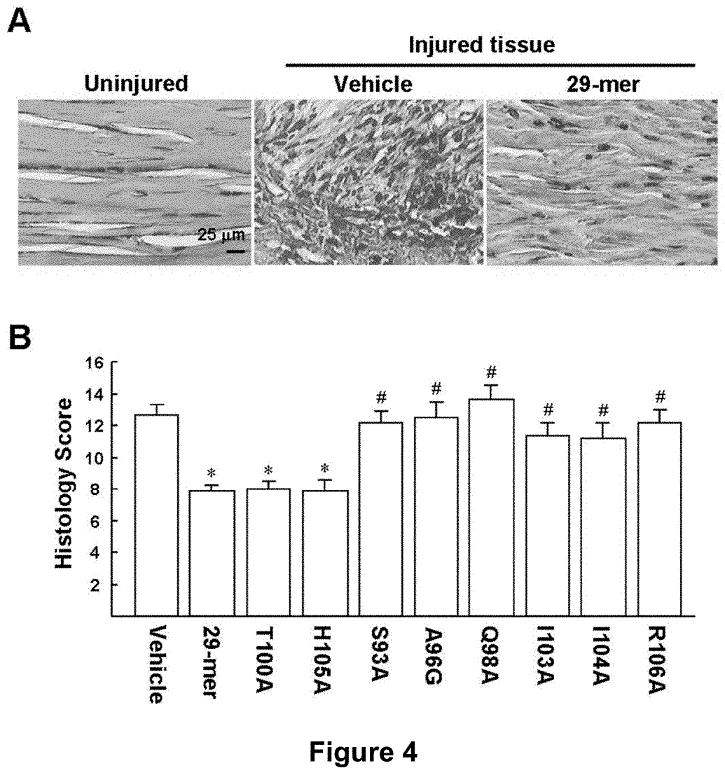

To investigate the therapeutic effect of the 29-mer peptide on injured tendon, a rat model of Achilles tendon injury was established by full-thickness insertion of an 18-G needle through the tendon Achilles. We delivered the 29-mer in a 150 μl alginate gel, which releases 90% loaded 29-mer in 5 days as described in a previous protocol (Ho et al., PEDF-derived peptide promotes skeletal muscle regeneration through its mitogenic effect on muscle progenitor cells. Am J Physiol. Cell Physiol. 2015 Aug. 1; 309(3): C159-68). As shown in FIG. 3, histological analysis by H&E staining at 1 week postoperative, vehicle/alginate gel treatment exhibited a loss of fiber organization and displayed a substantial presence of inflammatory matrix (marked by yellow arrows) and fatty deposits and/or necrotic area (marked by black arrows) in the healing region, whereas the 29-mer/alginate gel group showed a uniform appearance of well-aligned collagen fibers, indicating that the 29-mer promotes tendon healing. Notably, at 1 week postoperative, injured tendon treated with the vehicle group showed presence of a high number of rounded cells (fibroblast and inflammatory cells; FIG. 4A). In contrast, the 29-mer treatment displayed a great increase in cell density, and those nucleus morphologies showed a normal spindle-shaped tenocytes and slightly rounded resident cells disposed parallel to collagen fibers. Microscopically, injured tendon treated with alginate gel containing T100A or H105A variant also showed a uniform appearance of well-aligned collagen fibers and no degenerative events similar to the 29-mer treatment. Importantly, treatment with S93A, A96G, Q98A, 1103A, 1104A, and R106A showed a loss of fiber organization and accompanied a marked increase in inflammatory matrix, fatty deposits, and vascularity (marked by asterisk) in the tendon injured region (FIG. 3).

The scoring analysis was performed by two blinded examiners. The total histopathological scores are described above and presented in the histograms in FIG. 4B, the 29-mer/alginate treatment significantly reduced the total score, as compared with the vehicle/alginate group (7.9±0.4 versus 12.7±0.6; P<0.0005). T100A and H105A variants were also able to reduce total histopathological scores (8.0±0.5 and 7.8±0.7). Remarkably, treatment with S93A, A96G, Q98A, 1103A, 1104A, and R106A had no effect on the decrease in total histopathological scores (values among 11.2˜13.7), as compared with the 29-mer treatment.

Overall, the animal study results support that the 29-mer residues including S93, A96, Q98, 1103, 1104, and R106 are critical residues for sustaining the tendon therapeutic effect of 29-mer. These results also suggest that the core peptide with the tendon therapeutic effects is located at residues 93-106 of the human PEDF (⁹³SLGAEQRTESIIHR¹⁰⁶; SEQ ID NO:2). Therefore, in accordance with embodiments of the invention, a PDSP for the treatment of tendon injuries may be as short as a 14-mer (residues 93-106). One skilled in the art would appreciate that a peptide containing this core region would retain the same activities, even though additional amino acids may be included at the C and/or N terminus. That is a peptide for treating tendon injuries according to embodiments of the invention, may be a 14-mer, 15-mer, 16-mer, and so on, including the 29-mer used in the examples.

Furthermore, as noted above, substitutions within these short peptides can retain the activities, as long as the critical residues (serine-93, alanine-96, glutamine-98, isoleucine-103, isoleucine-104, and arginine-106) are preserved. In addition, the mouse variants (which have two substitutions: histidine-98 and valine-103, as compared with the human sequence) are also active. The corresponding mouse sequences are: mo-29mer (SLGAEHRTESVIHRALYYDLITNPDIHST, SEQ ID NO: 3) and mo-14mer (SLGAEHRTESVIHR, SEQID NO: 4). Thus, a generic sequence for an active core is (93S-X-X-A-X-Q/H-X-X-X-X-I/V-I-X-R¹⁰⁶, wherein X represents any amino-acid residue; SEQ ID NO: 5).

### Effects of the 29-Mer Variants on TSPC Spread at Damaged Tendon

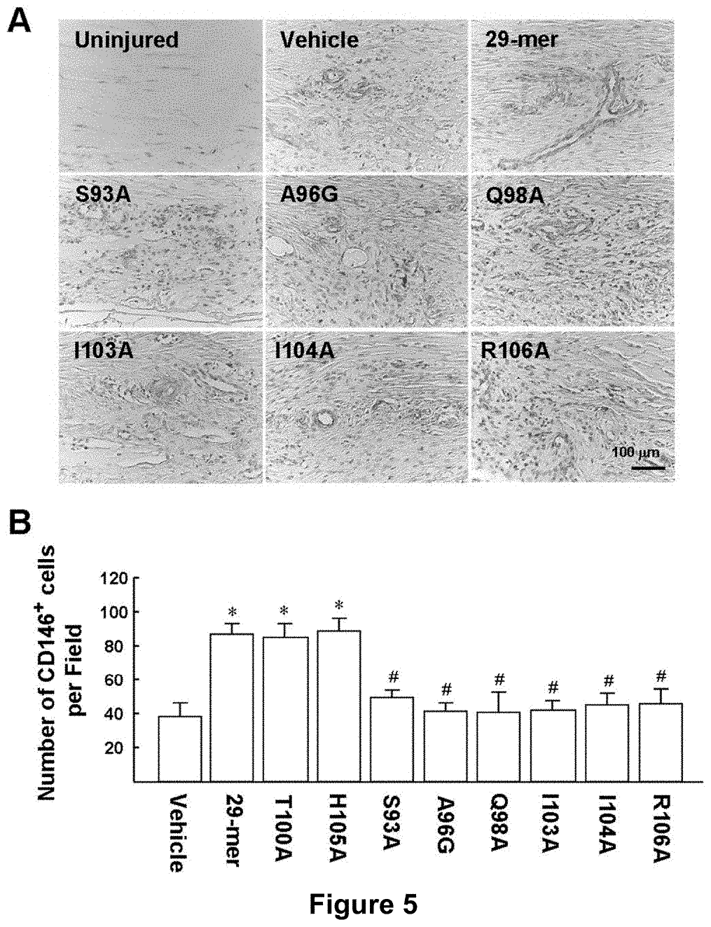

CD146 is one of the TSPC markers. CD146⁺ TSPC distributes at peripheral region of rat tendon and CD146⁺ TSPC has been found to assist wound healing of rat patellar tendon. To test the mechanism of the 29-mer-induced tendon repair, CD146 immunostaining of TSPC located at injured tendon was measured, at 1 week after wounding. The results revealed numerous CD146⁺ TSPC were detectable in the healing region of the 29-mer/alginate gel-treated tendons, whereas the vehicle/alginate gel-treated tendons had fewer CD146⁺ TSPC (FIG. 5; 86.8±6.0 versus 38.3±7.8 cells per 200× field). Thus, activation of TSPC expansion by the 29-mer treatment supports the speedy tendon wound healing.

Next, we examined the distribution of the CD146⁺ TSPC at damaged tendon treated with 29-mer variants for 1 week. The injured tendon treated with alginate gel containing T100A or H105A variant showed a significant CD146⁺ TSPC expansion (85.0±8.4 and 88.5±7.8 cells per 200× field) similar to the 29-mer/alginate treatment. Remarkably, CD146 immunostaining revealed treatment with S93A, A96G, Q98A, 1103A, 1104A, and R106A had no effect on the increase in CD146-positive TSPC at injured tendon (40.5˜49.5 cells per 200× field). The animal study further confirmed that those critical residues play crucial role for maintaining 29-mer biological activity.

Taken together, alanine scanning data indicate the therapeutic effect of the 29-mer is influenced by the amino acid substitution as evidenced by rat model of Achilles tendon rupture. Moreover, the 29-mer residues at positions S93, A96, Q98, 1103, 1104, and R106 are important for the 29-mer activity on tendon repair. Thus, a minimal core peptide may be represented as ⁹³S-X-X-A-X-Q/H-X-X-X-X-I/V-I-X-R¹⁰⁶, wherein X represents any amino-acid residue (SEQ ID NO:5). A few examples of PDSP sequence that may be used with embodiments of the invention are shown in the following Table (the positions numberings are based on the positions in the 14mers). These examples are not meant to be limiting.

| Peptide Sequences | SEQ ID NO |
|---|---|
| 1S-2X-3X-4A-5X-6Q/H-7X-8X-9X-10X-11I/V-12I-13X-14R | 5 |
| 1S-2L-3X-4A-5X-6Q/H-7X-8X-9X-10X-11I/V-12I-13X-14R | 6 |
| 1S-2A-3X-4A-5X-6Q/H-7X-8X-9X-10X-11I/V-12I-13X-14R | 7 |
| 1S-2X-3G-4A-5X-6Q/H-7X-8X-9X-10X-11I/V-12I-13X-14R | 8 |
| 1S-2X-3A-4A-5X-6Q/H-7X-8X-9X-10X-11I/V-12I 13X-14R | 9 |
| 1S-2X-3X-4A-5E-6Q/H-7X-8X-9X-10X-11I/V-I2I-13X-14R | 10 |
| 1S-2X-3X-4A-5A-6Q/H-7X-8X-9X-10X-11I/V-12I-13X-14R | 11 |
| 1S-2X-3X-4A-5X-6Q/H-7R-8X-9X-10X-11I/V-12I-13X-14R | 12 |
| 1S-2L-3X-4A-5X-6Q/H-7A-8X-9X-10X-11I/V-12I-13X-14R | 13 |
| 1S-2A-3X-4A-5X-6Q/H-7X-8T-9X-10X-11I/V-12I-13X-14R | 14 |
| 1S-2X-3G-4A-5X-6Q/H-7X-8A-9E-10X-11I/V-12I-13X-14R | 15 |
| 1S-2X-3A-4A-5X-6Q/H-7X-8X-9A-10X-11I/V-12I-13X-14R | 16 |
| 1S-2X-3X-4A-5E-6Q/H-7X-8X-9X-10S-11I/V-12I-13X-14R | 17 |
| 1S-2X-3X-4A-5A-6Q/H-7X-8X-9X-10A-11I/V-12I-13X-14R | 18 |
| 1S-2X-3X-4A-5X-6Q/H-7X-8X-9X-10X-11I/V-12I-13H-14R | 19 |
| 1S-2X-3X-4A-5X-6Q/H-7X-8X-9X-10X-11I/V-12I-13A-14R | 20 |
| 1S-2L-3G-4A-5X-6Q/H-7X-8X-9X-10X-11I/V-12I-13X-14R | 21 |
| 1S-2L-3G-4A-5E-6Q/H-7X-8X-9X-10X-11I/V-12I-13X-14R | 22 |
| 1S-2L-3G-4A-5A-6Q/H-7X-8X-9X-10X-11I/V-12I-13X-14R | 23 |
| 1S-2L-3G-4A-5X-6Q/H-7R-8X-9X-10X-11I/V-12I-13X-14R | 24 |
| 1S-2L-3G-4A-5X-6Q/H-7A-8X-9X-10X-11I/V-12I-13X-14R | 25 |
| 1S-2L-3G-4A-5X-gQ/H-7X-8T-9X-10X-11I/V-12I-13X-14R | 26 |
| 1S-2L-3G-4A-5X-6Q/H-7X-8A-9E-10X-11I/V-12I-13X-14R | 27 |
| 1S-2L-3G-4A-5X-6Q/H-7X-8X-9A-10X-11I/V-12I-13X-14R | 28 |
| 1S-2L-3G-4A-5X-6Q/H-7X-8X-9x-10S-11I/V-12I-13X-14R | 29 |
| 1S-2L-3G-4A-5X-6Q/H-7X-8X-9X-10A-11I/V-12I-13X-14R | 30 |
| 1S-2L-3G-4A-5X-6Q/H-7X-8X-9X-10S-11I/V-12I-13H-14R | 31 |
| 1S-2L-3G-4A-5X-6Q/H-7X-8X-9X-10A-11I/V-12I-13A-14R | 32 |
| 1S-2L-3G-4A-5E-6Q/H-7X-8X-9X-10X-11I/V-12I-13X-14R | 33 |
| 1S-2L-3G-4A-5E-6Q/H-7R-8X-9X-10X-11I/V-12I-13X-14R | 34 |
| 1S-2L-3G-4A-5E-6Q/H-7A-8X-9X-10X-11I/V-12I-13X-14R | 35 |
| 1S-2L-3G-4A-5E-6Q/H-7X-8T-9X-10X-11I/V-12I-13X-14R | 36 |
| 1S-2L-3G-4A-5E-6Q/H-7X-8A-9E-10X-11I/V-12I-13X-14R | 37 |
| 1S-2L-3G-4A-5E-6Q/H-7X-8X-9A-10X-11I/V-12I-13X-14R | 38 |
| 1S-2L-3G-4A-5E-6Q/H-7X-8X-9X-10S-11I/V-12I-13X 14R | 39 |
| 1S-2L-3G-4A-5E-6Q/H-7X-8X-9X-10A-11I/V-12I-13X-14R | 40 |
| 1S-2L-3G-4A-5E-6Q/H-7X-8X-9X-10S-11I/V-12I-13H-14R | 41 |
| 1S-2L-3G-4A-5E-6Q/H-7X-8X-9X-10A-11I/V-12I-13A-14R | 42 |
| 1S-2L-3G-4A-5E-6Q/H-7R-8X-9X-10X-11I/V-12I-13X-14R | 43 |
| 1S-2L-3G-4A-5E-6Q/H-7R-8T-9X-10X-11I/V-12I-13X-14R | 44 |
| 1S-2L-3G-4A-5E-6Q/H-7R-8A-9E-10X-11I/V-12I-13X-14R | 45 |
| 1S-2L-3G-4A-5E-6Q/H-7R-8X-9A-10X-11I/V-12I-13X-14R | 46 |
| 1S-2L-3G-4A-5E-6Q/H-7R-8X-9X-10S-11I/V-12I-13X-14R | 47 |
| 1S-2L-3G-4A-5E-6Q/H-7R-8X-9X-10A-11I/V-12I-13X-14R | 48 |
| 1S-2L-3G-4A-5E-6Q/H-7R-8X-9X-10S-11I/V-12I-13H-14R | 49 |
| 1S-2L-3G-4A-5E-6Q/H-7R-8X-9X-10A-11I/V-12I-13A-14R | 50 |
| 1S-2L-3G-4A-5E-6Q/H-7R-8T-9X-10X-11I/V-12I-13X-14R | 51 |
| 1S-2L-3G-4A-5E-6Q/H-7R-8T-9E-10X-11I/V-12I-13X-14R | 52 |
| 1S-2L-3G-4A-5E-6Q/H-7R-8T-9A-10X 11I/V-12I-13X-14R | 53 |
| 1S-2L-3G-4A-5E-6Q/H-7R-8T-9X-10S-11I/V-12I-13X-14R | 54 |
| 1S-2L-3G-4A-5E-6Q/H-7R-8T-9X-10A-11I/V-12I-13X-14R | 55 |
| 1S-2L-3G-4A-5E-6Q/H-7R-8T-9X-10S-11I/V-12I-13H-14R | 56 |
| 1S-2L-3G-4A-5E-6Q/H-7R-8T-9X-10A-11I/V-12I-13A-14R | 57 |
| 1S-2L-3G-4A-5E-6Q/H-7R-8T-9E-10S-11I/V-12I-13X-14R | 58 |
| 1S-2L-3G-4A-5E-6Q/H-7R-8T-9E-10A-11I/V-12I-13X-14R | 59 |
| 1S-2L-3G-4A-5E-6Q/H-7R-8T-9E-10X-11I/V-12I-13H-14R | 60 |
| 1S-2L-3G-4A-5E-6Q/H-7R-8T-9E-10X-11I/V-12I-13A-14R | 61 |
| 1S-2L-3G-4A-5E-6Q/H-7R-8T-9E-10S-11I/V-12I-13X-14R | 62 |
| 1S-2L-3G-4A-5E-6Q/H-7R-8T-9E-10S-11I/V-12I-13A-14R | 63 |
| 1S-2L-3G-4A-5E-6Q/H-7R-8T-9X-10S-11I/V-12I-13H-14R | 64 |
| 1S-2L-3G-4A-5E-6Q/H-7R-8T-9A-10S-11I/V-12I-13H-14R | 65 |
| 1S-2L-3G-4A-5E-6Q/H-7R-8X-9E-10S-11I/V-12I-13H-I4R | 66 |
| 1S-2L-3G-4A-5E-6Q/H-7R-8A-9E-10S-11I/V-12I-13H-14R | 67 |
| 1S-2L-3G-4A-5E-6Q/H-7X-8T-9E-10S-11I/V-12I-13H-14R | 68 |
| 1S-2L-3G-4A-5E-6Q/H-7A-8T-9E-10S-11I/V-12I-13H-14R | 69 |
| 1S-2L-3G-4A-5X-6Q/H-7R-8T-9E-10S-11I/V-12I-13H-14R | 70 |
| 1S-2L-3G-4A-5A-6Q/H-7R-8T-9E-10S-11I/V-12I-13H-14R | 71 |
| 1S-2L-3X-4A-5E-6Q/H-7R-8T-9E-10S-11I/V-12I-13H-14R | 72 |
| 1S-2L-3A-4A-5E-6Q/H-7R-8T-9E-10S-11I/V-12I-13H-14R | 73 |
| 1S-2X-3G-4A-5E-6Q/H-7R-8T-9E-10S-11I/V-12I-13H-14R | 74 |
| 1S-2A-3G-4A-5E-6Q/H-7R-8T-9E-10S-11I/V-12I-13H-14R | 75 |

Embodiments of the invention have been illustrated with a limited number of examples. One skilled in the art would appreciate that variations and modifications are possible without departing from the scope of the invention. Therefore, the scope of the invention should only be limited by the accompanied claims.

What is claimed is:

1. A pharmaceutical composition for use in treating a tendon injury, comprising: a PEDF-derived short peptide (PDSP) or a variant of the PDSP, wherein the PDSP comprises residues 93-106 of human pigmented epithelium-derived factor (PEDF), and wherein the variant of the PDSP contains serine-93, alanine-96, glutamine-98, isoleucine-103, isoleucine-104, and arginine 106 of the PDSP and contains one or more amino acid substitutions at other positions, wherein residue location numbers are based on those in the human PEDF.

2. The pharmaceutical composition according to claim 1, wherein the PDSP comprises the sequence of S-X-X-A-X-Q/H-X-X-X-X-I/V-I-X-R (SEQ ID NO:5).

3. The pharmaceutical composition according to claim 1, wherein the PDSP comprises the sequence of SLGAEQRTESIIHR (SEQ ID NO:2).

4. The pharmaceutical composition according to claim 1, wherein the PDSP comprises the sequence of SLGAEQRTESIIHRALYYDLISSPDIHGT (SEQ ID NO:1).

5. The pharmaceutical composition according to claim 1, wherein the PDSP comprises the sequence of the sequences of any one of SEQ ID NO: 6 to 75.

6. A method for treating a tendon injury, comprising: administering to a subject in need thereof a pharmaceutical composition comprising a PEDF-derived short peptide (PDSP) or a variant of the PDSP, wherein the PDSP comprises residues 93-106 of human pigmented epithelium-derived factor (PEDF), and wherein the variant of the PDSP contains serine-93, alanine-96, glutamine-98, isoleucine-103, isoleucine-104, and arginine 106 of the PDSP and contains one or more amino acid substitutions at other positions, wherein residue location numbers are based on those in the human PEDF.

7. The method according to claim 6, wherein the PDSP comprises the sequence of S-X-X-A-X-Q/H-X-X-X-X-I/V-I-X-R (SEQ ID NO:5).

8. The method according to claim 6, wherein the PDSP comprises the sequence of SLGAEQRTESIIHR (SEQ ID NO:2).

9. The method according to claim 6, wherein the PDSP comprises the sequence of SLGAEQRTESIIHRALYYDLISSPDIHGT (SEQ ID NO:1).

10. The method according to claim 6, wherein the PDSP comprises the sequence of the sequences of any one of SEQ ID NO: 6 to 75.


SEQUENCE LISTING (75 sequences)

SEQ ID NO: 1 - protein, 29 aa, Homo sapiens
SLGAEQRTESIIHRALYYDLISSPDIHGT

SEQ ID NO: 2 - protein, 14 aa, Homo sapiens
SLGAEQRTESIIHR

SEQ ID NO: 3 - protein, 29 aa, Mus musculus
SLGAEHRTESVIHRALYYDLITNPDIHST

SEQ ID NO: 4 - protein, 14 aa, Mus musculus
SLGAEHRTESVIHR

SEQ ID NO: 5 - protein, 14 aa, Artificial Sequence
  synthetic
  misc_feature (2)..(3): Xaa can be any naturally occurring amino acid
  misc_feature (5)..(11): Xaa can be any naturally occurring amino acid
  misc_feature (13)..(13): Xaa can be any naturally occurring amino acid
SXXAXXXXXXXIXR

SEQ ID NO: 6 - protein, 14 aa, Artificial Sequence
  synthetic
  misc_feature (3)..(3): Xaa can be any naturally occurring amino acid
  misc_feature (5)..(11): Xaa can be any naturally occurring amino acid
  misc_feature (13)..(13): Xaa can be any naturally occurring amino acid
SLXAXXXXXXXIXR

SEQ ID NO: 7 - protein, 14 aa, Artificial Sequence
  synthetic
  misc_feature (3)..(3): Xaa can be any naturally occurring amino acid
  misc_feature (5)..(11): Xaa can be any naturally occurring amino acid
  misc_feature (13)..(13): Xaa can be any naturally occurring amino acid
SAXAXXXXXXXIXR

SEQ ID NO: 8 - protein, 14 aa, Artificial Sequence
  synthetic
  misc_feature (2)..(2): Xaa can be any naturally occurring amino acid
  misc_feature (5)..(11): Xaa can be any naturally occurring amino acid
  misc_feature (13)..(13): Xaa can be any naturally occurring amino acid
SXGAXXXXXXXIXR

SEQ ID NO: 9 - protein, 14 aa, Artificial Sequence
  synthetic
  misc_feature (2)..(2): Xaa can be any naturally occurring amino acid
  misc_feature (5)..(11): Xaa can be any naturally occurring amino acid
  misc_feature (13)..(13): Xaa can be any naturally occurring amino acid
SXAAXXXXXXXIXR

SEQ ID NO: 10 - protein, 14 aa, Artificial Sequence
  synthetic
  misc_feature (2)..(3): Xaa can be any naturally occurring amino acid
  misc_feature (6)..(11): Xaa can be any naturally occurring amino acid
  misc_feature (13)..(13): Xaa can be any naturally occurring amino acid
SXXAEXXXXXXIXR

SEQ ID NO: 11 - protein, 14 aa, Artificial Sequence
  synthetic
  misc_feature (2)..(3): Xaa can be any naturally occurring amino acid
  misc_feature (6)..(11): Xaa can be any naturally occurring amino acid
  misc_feature (13)..(13): Xaa can be any naturally occurring amino acid
SXXAAXXXXXXIXR

SEQ ID NO: 12 - protein, 14 aa, Artificial Sequence
  synthetic
  misc_feature (2)..(3): Xaa can be any naturally occurring amino acid
  misc_feature (5)..(6): Xaa can be any naturally occurring amino acid
  misc_feature (8)..(11): Xaa can be any naturally occurring amino acid
  misc_feature (13)..(13): Xaa can be any naturally occurring amino acid
SXXAXXRXXXXIXR

SEQ ID NO: 13 - protein, 14 aa, Artificial Sequence
  synthetic
  misc_feature (2)..(3): Xaa can be any naturally occurring amino acid
  misc_feature (5)..(6): Xaa can be any naturally occurring amino acid
  misc_feature (8)..(11): Xaa can be any naturally occurring amino acid
  misc_feature (13)..(13): Xaa can be any naturally occurring amino acid
SXXAXXAXXXXIXR

SEQ ID NO: 14 - protein, 14 aa, Artificial Sequence
  synthetic
  misc_feature (2)..(3): Xaa can be any naturally occurring amino acid
  misc_feature (5)..(7): Xaa can be any naturally occurring amino acid
  misc_feature (9)..(11): Xaa can be any naturally occurring amino acid
  misc_feature (13)..(13): Xaa can be any naturally occurring amino acid
SXXAXXXTXXXIXR

SEQ ID NO: 15 - protein, 14 aa, Artificial Sequence
  synthetic
  misc_feature (2)..(3): Xaa can be any naturally occurring amino acid
  misc_feature (5)..(7): Xaa can be any naturally occurring amino acid
  misc_feature (10)..(11): Xaa can be any naturally occurring amino acid
  misc_feature (13)..(13): Xaa can be any naturally occurring amino acid
SXXAXXXAEXXIXR

SEQ ID NO: 16 - protein, 14 aa, Artificial Sequence
  synthetic
  misc_feature (2)..(3): Xaa can be any naturally occurring amino acid
  misc_feature (5)..(8): Xaa can be any naturally occurring amino acid
  misc_feature (10)..(11): Xaa can be any naturally occurring amino acid
  misc_feature (13)..(13): Xaa can be any naturally occurring amino acid
SXXAXXXXAXXIXR

SEQ ID NO: 17 - protein, 14 aa, Artificial Sequence
  synthetic
  misc_feature (2)..(3): Xaa can be any naturally occurring amino acid
  misc_feature (5)..(9): Xaa can be any naturally occurring amino acid
  misc_feature (11)..(11): Xaa can be any naturally occurring amino acid
  misc_feature (13)..(13): Xaa can be any naturally occurring amino acid
SXXAXXXXXSXIXR

SEQ ID NO: 18 - protein, 14 aa, Artificial Sequence
  synthetic
  misc_feature (2)..(3): Xaa can be any naturally occurring amino acid
  misc_feature (5)..(9): Xaa can be any naturally occurring amino acid
  misc_feature (11)..(11): Xaa can be any naturally occurring amino acid
  misc_feature (13)..(13): Xaa can be any naturally occurring amino acid
SXXAXXXXXAXIXR

SEQ ID NO: 19 - protein, 14 aa, Artificial Sequence
  synthetic
  misc_feature (2)..(3): Xaa can be any naturally occurring amino acid
  misc_feature (5)..(11): Xaa can be any naturally occurring amino acid
SXXAXXXXXXXIHR

SEQ ID NO: 20 - protein, 14 aa, Artificial Sequence
  synthetic
  misc_feature (2)..(3): Xaa can be any naturally occurring amino acid
  misc_feature (5)..(11): Xaa can be any naturally occurring amino acid
SXXAXXXXXXXIAR

SEQ ID NO: 21 - protein, 14 aa, Artificial Sequence
  synthetic
  misc_feature (5)..(11): Xaa can be any naturally occurring amino acid
  misc_feature (13)..(13): Xaa can be any naturally occurring amino acid
SLGAXXXXXXXIXR

SEQ ID NO: 22 - protein, 14 aa, Artificial Sequence
  synthetic
  misc_feature (6)..(11): Xaa can be any naturally occurring amino acid
  misc_feature (13)..(13): Xaa can be any naturally occurring amino acid
SLGAEXXXXXXIXR

SEQ ID NO: 23 - protein, 14 aa, Artificial Sequence
  synthetic
  misc_feature (6)..(11): Xaa can be any naturally occurring amino acid
  misc_feature (13)..(13): Xaa can be any naturally occurring amino acid
SLGAAXXXXXXIXR

SEQ ID NO: 24 - protein, 14 aa, Artificial Sequence
  synthetic
  misc_feature (5)..(6): Xaa can be any naturally occurring amino acid
  misc_feature (8)..(11): Xaa can be any naturally occurring amino acid
  misc_feature (13)..(13): Xaa can be any naturally occurring amino acid
SLGAXXRXXXXIXR

SEQ ID NO: 25 - protein, 14 aa, Artificial Sequence
  synthetic
  misc_feature (5)..(6): Xaa can be any naturally occurring amino acid
  misc_feature (8)..(11): Xaa can be any naturally occurring amino acid
  misc_feature (13)..(13): Xaa can be any naturally occurring amino acid
SLGAXXAXXXXIXR

SEQ ID NO: 26 - protein, 14 aa, Artificial Sequence
  synthetic
  misc_feature (5)..(7): Xaa can be any naturally occurring amino acid
  misc_feature (9)..(11): Xaa can be any naturally occurring amino acid
  misc_feature (13)..(13): Xaa can be any naturally occurring amino acid
SLGAXXXTXXXIXR

SEQ ID NO: 27 - protein, 14 aa, Artificial Sequence
  synthetic
  misc_feature (5)..(7): Xaa can be any naturally occurring amino acid
  misc_feature (10)..(11): Xaa can be any naturally occurring amino acid
  misc_feature (13)..(13): Xaa can be any naturally occurring amino acid
SLGAXXXAEXXIXR

SEQ ID NO: 28 - protein, 14 aa, Artificial Sequence
  synthetic
  misc_feature (5)..(8): Xaa can be any naturally occurring amino acid
  misc_feature (10)..(11): Xaa can be any naturally occurring amino acid
  misc_feature (13)..(13): Xaa can be any naturally occurring amino acid
SLGAXXXXAXXIXR

SEQ ID NO: 29 - protein, 14 aa, Artificial Sequence
  synthetic
  misc_feature (5)..(9): Xaa can be any naturally occurring amino acid
  misc_feature (11)..(11): Xaa can be any naturally occurring amino acid
  misc_feature (13)..(13): Xaa can be any naturally occurring amino acid
SLGAXXXXXSXIXR

SEQ ID NO: 30 - protein, 14 aa, Artificial Sequence
  synthetic
  misc_feature (5)..(9): Xaa can be any naturally occurring amino acid
  misc_feature (11)..(11): Xaa can be any naturally occurring amino acid
  misc_feature (13)..(13): Xaa can be any naturally occurring amino acid
SLGAXXXXXAXIXR

SEQ ID NO: 31 - protein, 14 aa, Artificial Sequence
  synthetic
  misc_feature (5)..(9): Xaa can be any naturally occurring amino acid
  misc_feature (11)..(11): Xaa can be any naturally occurring amino acid
SLGAXXXXXSXIHR

SEQ ID NO: 32 - protein, 14 aa, Artificial Sequence
  synthetic
  misc_feature (5)..(9): Xaa can be any naturally occurring amino acid
  misc_feature (11)..(11): Xaa can be any naturally occurring amino acid
SLGAXXXXXAXIAR

SEQ ID NO: 33 - protein, 14 aa, Artificial Sequence
  synthetic
  misc_feature (6)..(11): Xaa can be any naturally occurring amino acid
  misc_feature (13)..(13): Xaa can be any naturally occurring amino acid
SLGAEXXXXXXIXR

SEQ ID NO: 34 - protein, 14 aa, Artificial Sequence
  synthetic
  misc_feature (6)..(6): Xaa can be any naturally occurring amino acid
  misc_feature (8)..(11): Xaa can be any naturally occurring amino acid
  misc_feature (13)..(13): Xaa can be any naturally occurring amino acid
SLGAEXRXXXXIXR

SEQ ID NO: 35 - protein, 14 aa, Artificial Sequence
  synthetic
  misc_feature (6)..(6): Xaa can be any naturally occurring amino acid
  misc_feature (8)..(11): Xaa can be any naturally occurring amino acid
  misc_feature (13)..(13): Xaa can be any naturally occurring amino acid
SLGAEXAXXXXIXR

SEQ ID NO: 36 - protein, 14 aa, Artificial Sequence
  synthetic
  misc_feature (6)..(7): Xaa can be any naturally occurring amino acid
  misc_feature (9)..(11): Xaa can be any naturally occurring amino acid
  misc_feature (13)..(13): Xaa can be any naturally occurring amino acid
SLGAEXXTXXXIXR

SEQ ID NO: 37 - protein, 14 aa, Artificial Sequence
  synthetic
  misc_feature (6)..(7): Xaa can be any naturally occurring amino acid
  misc_feature (10)..(11): Xaa can be any naturally occurring amino acid
  misc_feature (13)..(13): Xaa can be any naturally occurring amino acid
SLGAEXXAEXXIXR

SEQ ID NO: 38 - protein, 14 aa, Artificial Sequence
  synthetic
  misc_feature (6)..(8): Xaa can be any naturally occurring amino acid
  misc_feature (10)..(11): Xaa can be any naturally occurring amino acid
  misc_feature (13)..(13): Xaa can be any naturally occurring amino acid
SLGAEXXXAXXIXR

SEQ ID NO: 39 - protein, 14 aa, Artificial Sequence
  synthetic
  misc_feature (6)..(9): Xaa can be any naturally occurring amino acid
  misc_feature (11)..(11): Xaa can be any naturally occurring amino acid
  misc_feature (13)..(13): Xaa can be any naturally occurring amino acid
SLGAEXXXXSXIXR

SEQ ID NO: 40 - protein, 14 aa, Artificial Sequence
  synthetic
  misc_feature (6)..(9): Xaa can be any naturally occurring amino acid
  misc_feature (11)..(11): Xaa can be any naturally occurring amino acid
  misc_feature (13)..(13): Xaa can be any naturally occurring amino acid
SLGAEXXXXAXIXR

SEQ ID NO: 41 - protein, 14 aa, Artificial Sequence
  synthetic
  misc_feature (6)..(9): Xaa can be any naturally occurring amino acid
  misc_feature (11)..(11): Xaa can be any naturally occurring amino acid
SLGAEXXXXSXIHR

SEQ ID NO: 42 - protein, 14 aa, Artificial Sequence
  synthetic
  misc_feature (6)..(9): Xaa can be any naturally occurring amino acid
  misc_feature (11)..(11): Xaa can be any naturally occurring amino acid
SLGAEXXXXAXIAR

SEQ ID NO: 43 - protein, 14 aa, Artificial Sequence
  synthetic
  misc_feature (6)..(6): Xaa can be any naturally occurring amino acid
  misc_feature (8)..(11): Xaa can be any naturally occurring amino acid
  misc_feature (13)..(13): Xaa can be any naturally occurring amino acid
SLGAEXRXXXXIXR

SEQ ID NO: 44 - protein, 14 aa, Artificial Sequence
  synthetic
  misc_feature (6)..(6): Xaa can be any naturally occurring amino acid
  misc_feature (9)..(11): Xaa can be any naturally occurring amino acid
  misc_feature (13)..(13): Xaa can be any naturally occurring amino acid
SLGAEXRTXXXIXR

SEQ ID NO: 45 - protein, 14 aa, Artificial Sequence
  synthetic
  misc_feature (6)..(6): Xaa can be any naturally occurring amino acid
  misc_feature (9)..(11): Xaa can be any naturally occurring amino acid
  misc_feature (13)..(13): Xaa can be any naturally occurring amino acid
SLGAEXRAXXXIXR

SEQ ID NO: 46 - protein, 14 aa, Artificial Sequence
  synthetic
  misc_feature (6)..(6): Xaa can be any naturally occurring amino acid
  misc_feature (8)..(8): Xaa can be any naturally occurring amino acid
  misc_feature (10)..(11): Xaa can be any naturally occurring amino acid
  misc_feature (13)..(13): Xaa can be any naturally occurring amino acid
SLGAEXRXAXXIXR

SEQ ID NO: 47 - protein, 14 aa, Artificial Sequence
  synthetic
  misc_feature (6)..(6): Xaa can be any naturally occurring amino acid
  misc_feature (8)..(9): Xaa can be any naturally occurring amino acid
  misc_feature (11)..(11): Xaa can be any naturally occurring amino acid
  misc_feature (13)..(13): Xaa can be any naturally occurring amino acid
SLGAEXRXXSXIXR

SEQ ID NO: 48 - protein, 14 aa, Artificial Sequence
  synthetic
  misc_feature (6)..(6): Xaa can be any naturally occurring amino acid
  misc_feature (8)..(9): Xaa can be any naturally occurring amino acid
  misc_feature (11)..(11): Xaa can be any naturally occurring amino acid
  misc_feature (13)..(13): Xaa can be any naturally occurring amino acid
SLGAEXRXXAXIXR

SEQ ID NO: 49 - protein, 14 aa, Artificial Sequence
  synthetic
  misc_feature (6)..(6): Xaa can be any naturally occurring amino acid
  misc_feature (8)..(9): Xaa can be any naturally occurring amino acid
  misc_feature (11)..(11): Xaa can be any naturally occurring amino acid
SLGAEXRXXSXIHR

SEQ ID NO: 50 - protein, 14 aa, Artificial Sequence
  synthetic
  misc_feature (6)..(6): Xaa can be any naturally occurring amino acid
  misc_feature (8)..(9): Xaa can be any naturally occurring amino acid
  misc_feature (11)..(11): Xaa can be any naturally occurring amino acid
SLGAEXRXXAXIAR

SEQ ID NO: 51 - protein, 14 aa, Artificial Sequence
  synthetic
  misc_feature (6)..(6): Xaa can be any naturally occurring amino acid
  misc_feature (9)..(11): Xaa can be any naturally occurring amino acid
  misc_feature (13)..(13): Xaa can be any naturally occurring amino acid
SLGAEXRTXXXIXR

SEQ ID NO: 52 - protein, 14 aa, Artificial Sequence
  synthetic
  misc_feature (6)..(6): Xaa can be any naturally occurring amino acid
  misc_feature (10)..(11): Xaa can be any naturally occurring amino acid
  misc_feature (13)..(13): Xaa can be any naturally occurring amino acid
SLGAEXRTEXXIXR

SEQ ID NO: 53 - protein, 14 aa, Artificial Sequence
  synthetic
  misc_feature (6)..(6): Xaa can be any naturally occurring amino acid
  misc_feature (10)..(11): Xaa can be any naturally occurring amino acid
  misc_feature (13)..(13): Xaa can be any naturally occurring amino acid
SLGAEXRTAXXIXR

SEQ ID NO: 54 - protein, 14 aa, Artificial Sequence
  synthetic
  misc_feature (6)..(6): Xaa can be any naturally occurring amino acid
  misc_feature (9)..(9): Xaa can be any naturally occurring amino acid
  misc_feature (11)..(11): Xaa can be any naturally occurring amino acid
  misc_feature (13)..(13): Xaa can be any naturally occurring amino acid
SLGAEXRTXSXIXR

SEQ ID NO: 55 - protein, 14 aa, Artificial Sequence
  synthetic
  misc_feature (6)..(6): Xaa can be any naturally occurring amino acid
  misc_feature (9)..(9): Xaa can be any naturally occurring amino acid
  misc_feature (11)..(11): Xaa can be any naturally occurring amino acid
  misc_feature (13)..(13): Xaa can be any naturally occurring amino acid
SLGAEXRTXAXIXR

SEQ ID NO: 56 - protein, 14 aa, Artificial Sequence
  synthetic
  misc_feature (6)..(6): Xaa can be any naturally occurring amino acid
  misc_feature (9)..(9): Xaa can be any naturally occurring amino acid
  misc_feature (11)..(11): Xaa can be any naturally occurring amino acid
SLGAEXRTXSXIHR

SEQ ID NO: 57 - protein, 14 aa, Artificial Sequence
  synthetic
  misc_feature (6)..(6): Xaa can be any naturally occurring amino acid
  misc_feature (9)..(9): Xaa can be any naturally occurring amino acid
  misc_feature (11)..(11): Xaa can be any naturally occurring amino acid
SLGAEXRTXAXIAR

SEQ ID NO: 58 - protein, 14 aa, Artificial Sequence
  synthetic
  misc_feature (6)..(6): Xaa can be any naturally occurring amino acid
  misc_feature (11)..(11): Xaa can be any naturally occurring amino acid
  misc_feature (13)..(13): Xaa can be any naturally occurring amino acid
SLGAEXRTESXIXR

SEQ ID NO: 59 - protein, 14 aa, Artificial Sequence
  synthetic
  misc_feature (6)..(6): Xaa can be any naturally occurring amino acid
  misc_feature (11)..(11): Xaa can be any naturally occurring amino acid
  misc_feature (13)..(13): Xaa can be any naturally occurring amino acid
SLGAEXRTEAXIXR

SEQ ID NO: 60 - protein, 14 aa, Artificial Sequence
  synthetic
  misc_feature (6)..(6): Xaa can be any naturally occurring amino acid
  misc_feature (10)..(11): Xaa can be any naturally occurring amino acid
SLGAEXRTEXXIHR

SEQ ID NO: 61 - protein, 14 aa, Artificial Sequence
  synthetic
  misc_feature (6)..(6): Xaa can be any naturally occurring amino acid
  misc_feature (10)..(11): Xaa can be any naturally occurring amino acid
SLGAEXRTEXXIAR

SEQ ID NO: 62 - protein, 14 aa, Artificial Sequence
  synthetic
  misc_feature (6)..(6): Xaa can be any naturally occurring amino acid
  misc_feature (11)..(11): Xaa can be any naturally occurring amino acid
  misc_feature (13)..(13): Xaa can be any naturally occurring amino acid
SLGAEXRTESXIXR

SEQ ID NO: 63 - protein, 14 aa, Artificial Sequence
  synthetic
  misc_feature (6)..(6): Xaa can be any naturally occurring amino acid
  misc_feature (11)..(11): Xaa can be any naturally occurring amino acid
SLGAEXRTESXIAR

SEQ ID NO: 64 - protein, 14 aa, Artificial Sequence
  synthetic
  misc_feature (6)..(6): Xaa can be any naturally occurring amino acid
  misc_feature (9)..(9): Xaa can be any naturally occurring amino acid
  misc_feature (11)..(11): Xaa can be any naturally occurring amino acid
SLGAEXRTXSXIHR

SEQ ID NO: 65 - protein, 14 aa, Artificial Sequence
  synthetic
  misc_feature (6)..(6): Xaa can be any naturally occurring amino acid
  misc_feature (11)..(11): Xaa can be any naturally occurring amino acid
SLGAEXRTASXIHR

SEQ ID NO: 66 - protein, 14 aa, Artificial Sequence
  synthetic
  misc_feature (6)..(6): Xaa can be any naturally occurring amino acid
  misc_feature (8)..(8): Xaa can be any naturally occurring amino acid
  misc_feature (11)..(11): Xaa can be any naturally occurring amino acid
SLGAEXRXESXIHR

SEQ ID NO: 67 - protein, 14 aa, Artificial Sequence
  synthetic
  misc_feature (6)..(6): Xaa can be any naturally occurring amino acid
  misc_feature (11)..(11): Xaa can be any naturally occurring amino acid
SLGAEXRAESXIHR

SEQ ID NO: 68 - protein, 14 aa, Artificial Sequence
  synthetic
  misc_feature (6)..(7): Xaa can be any naturally occurring amino acid
  misc_feature (11)..(11): Xaa can be any naturally occurring amino acid
SLGAEXXTESXIHR

SEQ ID NO: 69 - protein, 14 aa, Artificial Sequence
  synthetic
  misc_feature (6)..(6): Xaa can be any naturally occurring amino acid
  misc_feature (11)..(11): Xaa can be any naturally occurring amino acid
SLGAEXATESXIHR

SEQ ID NO: 70 - protein, 14 aa, Artificial Sequence
  synthetic
  misc_feature (5)..(6): Xaa can be any naturally occurring amino acid
  misc_feature (11)..(11): Xaa can be any naturally occurring amino acid
SLGAXXRTESXIHR

SEQ ID NO: 71 - protein, 14 aa, Artificial Sequence
  synthetic
  misc_feature (6)..(6): Xaa can be any naturally occurring amino acid
  misc_feature (11)..(11): Xaa can be any naturally occurring amino acid
SLGAAXRTESXIHR

SEQ ID NO: 72 - protein, 14 aa, Artificial Sequence
  synthetic
  misc_feature (3)..(3): Xaa can be any naturally occurring amino acid
  misc_feature (6)..(6): Xaa can be any naturally occurring amino acid
  misc_feature (11)..(11): Xaa can be any naturally occurring amino acid
SLXAEXRTESXIHR

SEQ ID NO: 73 - protein, 14 aa, Artificial Sequence
  synthetic
  misc_feature (6)..(6): Xaa can be any naturally occurring amino acid
  misc_feature (11)..(11): Xaa can be any naturally occurring amino acid
SLAAEXRTESXIHR

SEQ ID NO: 74 - protein, 14 aa, Artificial Sequence
  synthetic
  misc_feature (2)..(2): Xaa can be any naturally occurring amino acid
  misc_feature (6)..(6): Xaa can be any naturally occurring amino acid
  misc_feature (11)..(11): Xaa can be any naturally occurring amino acid
SXGAEXRTESXIHR

SEQ ID NO: 75 - protein, 14 aa, Artificial Sequence
  synthetic
  misc_feature (6)..(6): Xaa can be any naturally occurring amino acid
  misc_feature (11)..(11): Xaa can be any naturally occurring amino acid
SAGAEXRTESXIHR